(19) United States
(12) Patent Application Publication
(10) Pub. No.: US 2006/0269915 A1
(43) Pub. Date: Nov. 30, 2006

(54) Pyrophosphate acid detection sensor, method of detection of nucleic acid, and method of discrimination of base type

(75) Inventor: Tetsuo Yukimasa, Nara-shi, Nara (JP)

Correspondence Address: MCDERMOTT WILL & EMERY LLP, 600 13TH STREET, N.W., WASHINGTON, DC, 20005-3096, US

(73) Assignee: Matsushita Electric Industrial Co., Ltd., Osaka (JP)

(21) Appl. No.: 10/536,014

(22) PCT Filed: Jul. 2, 2004
(86) PCT No.: PCT/JP04/09787

§ 371 Date: May 23, 2005

(30) Foreign Application Priority Data
Jul. 2, 2003 (JP) 2003-190121

(51) Int. Cl. C12Q 1/68; C12M 1/34

(52) U.S. Cl. 435/6; 435/287.2

(57) ABSTRACT

The present invention is a pyrophosphate detection sensor for detecting pyrophosphate in a sample solution, and the sensor is constituted to have a sample solution receptive part that receives the sample solution, a H⁺ impermeable membrane having H⁺-pyrophosphatase, an immobilizing layer for immobilizing the H⁺ impermeable membrane, and a measurement means for measuring an electrochemical alteration with the change in hydrogen ion concentration of the immobilizing layer. The H⁺-pyrophosphatase is provided such that it hydrolyzes pyrophosphate in the sample solution to result in change in the hydrogen ion concentration of the immobilizing layer.

### TECHNICAL FIELD

The present invention relates to a sensor for detecting pyrophosphate in a sample readily and with high sensitivity; and a method of the detection of a nucleic acid and a method of the discrimination of a base type using the same.

### BACKGROUND ART

Pyrophosphate has been known to greatly participate in the enzymatic reaction within a cell. For example, in a synthetic process of a protein, the pyrophosphate is produced in a reaction to form aminoacyl tRNA from an amino acid via aminoacyl adenylate. Also, in a synthetic process of starch found in, for example, plants and the like, the pyrophosphate is produced by a reaction of glucose-l-phosphate and ATP to produce ADP-glucose. In addition, pyrophosphate has been known to participate in a variety of enzymatic reactions. Therefore, techniques to quantitatively detect the pyrophosphate are important upon analyses of the cellular states, the aforementioned enzymatic reactions and the like.

As a conventional method of the measurement of pyrophosphate, a chemical method of Grindley et al., (see, G. B. Grindley and C. A. Nichel, Anal. Biochem, vol. 33. p. 114 (1970)) has been known. However, great risk is involved in this method because concentrated sulfuric acid is used.

JP-A-61-12300 discloses three kinds of methods of the measurement of pyrophosphate utilizing an enzyme without using a dangerous agent such as concentrated sulfuric acid. These methods are explained below.

First method involves subjecting the pyrophosphate to an action of pyruvate orthophosphate dikinase in the presence of phosphoenol pyruvate and adenosine monophosphate. Pyruvic acid is formed by this reaction, therefore, the amount of the pyrophosphate can be calculated by measuring the amount of pyruvic acid. For reference, two kinds of methods of the measurement of the amount of pyruvic acid have been proposed. One is a method to conduct colorimetric determination of decrease in NADH upon reduction of pyruvic acid with NADH utilizing a catalytic action of lactate dehydrogenase. Another is a method to conduct colorimetric determination by derivatizing a dye stuff from hydrogen peroxide which is produced through subjecting the produced pyruvic acid to an action of pyruvate oxidase.

Second method involves subjecting the pyrophosphate to an action of glycerol-3-phosphate cytidyl transferase in the presence of cytidine diphosphoglycerol. According to this reaction, glycerol triphosphate is produced. Therefore, measurement of the amount of production of glycerol triphosphate enables the calculation of the amount of the pyrophosphate. Two kinds of methods of the measurement of the amount of glycerol triphosphate have been proposed. One is a method to conduct calorimetric determination of increase in NAD(P)H upon oxidation of glycerol triphosphate with NAD(P) utilizing a catalytic action of glycerol-3-phosphate dehydrogenase. Another is a method to conduct calorimetric determination by derivatizing a dye stuff from hydrogen peroxide which is produced through subjecting the produced glycerol triphosphate to an action of glycerol-3-phosphate oxidase.

Third method involves subjecting the pyrophosphate to an action of ribitol-5-phosphate cytidyltransferase in the presence of cytidine diphosphate ribitol. According to this reaction, D-ribitol-5-phosphate is produced, whereby enabling the measurement of the amount of the pyrophosphate through measuring the amount of production. As a method of the measurement of D-ribitol-5-phosphate, a method to conduct calorimetric determination of increase in NADH (or NADPH) has been proposed through allowing ribitol-5-phosphate dehydrogenase to act in the presence of NAD (or NADP).

In addition, JP-A-2002-369698 discloses a method comprising: hydrolyzing the pyrophosphate into phosphoric acid by pyrophosphatase; allowing the phosphoric acid to react with inosine or xanthosine by purine nucleoside phosphorylase; oxidizing thus resulting hypoxanthine by xanthine oxidase to give xanthine; further oxidizing the product to produce uric acid; and allowing coloring of a coloring agent utilizing peroxidase with hydrogen peroxide generated during this oxidation step by this xanthine oxidase.

On the other hand, extension reactions of nucleic acids are also one of important biological reactions in which pyrophosphate is involved.

In recent years, techniques in connection with gene information have been extensively developed. In medical field, therapies of diseases at a molecular level have been enabled through analyzing disease-related genes. Furthermore, gene diagnoses have enabled tailor-made therapies corresponding to individual patients. In pharmaceutical field, protein molecules such as antibodies and hormones have been specified using gene information, which have been utilized as medicaments. Also in agricultural or food field, many products in which gene information is utilized have been created.

Among the gene information, gene polymorphisms are of particular importance. As our faces and body types vary from person to person, the individual gene information varies in considerable segments. Among these differences in genetic information, those with the alteration of the base sequence being present with an incidence of 1% or greater on a population basis are referred to as gene polymorphism. Such gene polymorphisms are referred to as being related not only to individual facial appearances, but also to a variety of causes of hereditary diseases, constitutions, responsiveness to a drug, side effects of a drug and the like. Therefore, relationship between the gene polymorphism and diseases has been rapidly investigated at present.

Among these gene polymorphisms, those particularly attracting attention in recent years are SNPs (Single nucleotide polymorphism). SNP indicates a gene polymorphism including a difference in only one base in a base sequence of the gene information. SNPs are referred to as being in existence by 2 to 3 million within human genomic DNAs, can be readily utilized as a marker for a gene polymorphism, and have been expected for clinical applications. At present, in connection with SNP-related techniques, identification of the location of SNP within a genome and investigations of relationships between a SNP and a disease and the like as well as development of SNP typing techniques to discriminate the base in a SNP site have been carried out.

There are various kinds of techniques of the SNP typing such as those in which hybridization is utilized, in which a restriction enzyme is utilized, in which an enzyme such as ligase is utilized, and the like. Among those techniques, the most convenient technique utilizes a primer extension reaction. According to this technique, the SNP typing is carried out by determining as to whether the primer extension reaction occurs.

For the detection in SNP typing techniques in which a primer extension reaction is utilized, there have been proposed a method of the detection of amplified products of an actual DNA using a fluorescent dye stuff, a method in which an immobilized probe is used, and also a method of the detection of pyrophosphate which is a byproduct of the nucleic acid synthesis by DNA polymerase. According to this method, in order to detect the difference in progress of extension reactions, a process is employed wherein the pyrophosphate produced with the progress of the primer extension reaction is converted into ATP, and thereafter, the amount of the pyrophosphate is measured utilizing a luciferase reaction (see, J. Immunological Method, 156, 55-60, 1992).

However, in the method of the measurement of pyrophosphate according to the aforementioned process, dATP becomes the substrate for the luciferase reaction similarly to ATP, when dATP is used in the primer extension reaction. Thus, the base type of the SNP site can not be accurately discriminated. Accordingly, there have existed problems that a particular dATP analogue must be used which acts as a substrate for DNA polymerase in stead of dATP, but does not act as a substrate for a luciferase reaction.

Also, even in cases of other measurement techniques of pyrophosphate, there have existed disadvantages that multiple kinds of enzymes, reagents and the like are required, whereby increasing the cost and complicating the steps. Also, according to these methods of measurement, there have existed problems of difficulties in making a kit and making a device as a compact sensor, on the grounds described above.

Furthermore, because almost of these methods are optical detection methods, there have also existed problems of gaining in size of the measurement apparatus.

### DISCLOSURE OF THE INVENTION

The present invention was made in order to solve the problems described above, and an object of the invention is to provide a pyrophosphate detection sensor for detecting pyrophosphate in a sample solution readily with high sensitivity, having a simple construction; and a method of the detection of a nucleic acid and a method of the discrimination of a base type using the same.

In order to accomplish the object, the present invention provides a pyrophosphate detection sensor for detecting pyrophosphate in a sample solution, which comprises: a sample solution receptive part that receives the aforementioned sample solution, a H⁺ impermeable membrane having H⁺-pyrophosphatase, an immobilizing layer for immobilizing the aforementioned H⁺ impermeable membrane, and a measurement means for measuring an electrochemical alteration with the change in hydrogen ion concentration of the aforementioned immobilizing layer, wherein the aforementioned H⁺-pyrophosphatase is provided such that it hydrolyzes the pyrophosphate in the aforementioned sample solution to result in change in the hydrogen ion concentration of the immobilizing layer. According to such a constitution, the immobilizing layer in which change in the concentration of the hydrogen ion is caused by the H⁺-pyrophosphatase also has a function to immobilize the H⁺ impermeable membrane, therefore, a pyrophosphate detection sensor can be conveniently configured.

One mode of the pyrophosphate detection sensor described above has a constitution in which the aforementioned immobilizing layer immobilizes the H⁺ impermeable membrane on the upper face or inside thereof.

One mode of the pyrophosphate detection sensor described above has a constitution in which the aforementioned H⁺ impermeable membrane is a membrane vesicle, and the aforementioned immobilizing layer immobilizes the aforementioned H⁺ impermeable membrane inside thereof.

One mode of the pyrophosphate detection sensor described above has a constitution in which the aforementioned measurement means has a hydrogen ion sensitive electrode which is in contact with the aforementioned immobilizing layer, and a reference electrode provided such that it is in contact with the aforementioned sample solution in the state of receiving the aforementioned sample solution. Further, the aforementioned measurement means is preferably configured such that an alteration of the electric potential difference between the aforementioned hydrogen ion sensitive electrode and the aforementioned reference electrode is measured.

One mode of the pyrophosphate detection sensor described above has a constitution in which the aforementioned immobilizing layer comprises a polymer gel or a self-assembled monomolecular membrane (hereinafter, also referred to as SAM membrane). The polymer gel can immobilize the aforementioned H⁺ impermeable membrane due to its holding capacity.

One mode of the pyrophosphate detection sensor described above has a constitution in which the aforementioned immobilizing layer comprises a material which causes an oxidation reduction reaction with change in the hydrogen ion concentration, and the aforementioned measurement means has a polarizable electrode that is in contact with the aforementioned immobilizing layer, and a reference electrode provided such that it is in contact with the aforementioned sample solution in the state of receiving the aforementioned sample solution. Further, the aforementioned measurement means is preferably configured such that an alteration of the electric current between the aforementioned polarizable electrode and the aforementioned reference electrode is measured.

One mode of the pyrophosphate detection sensor described above has a constitution in which the aforementioned immobilizing layer comprises a polymer gel containing a mediator which causes an oxidation reduction reaction with change in the hydrogen ion concentration, or a self-assembled monomolecular membrane, and the aforementioned H⁺ impermeable membrane is immobilized on the upper face thereof.

One mode of the pyrophosphate detection sensor described above has a constitution in which the aforementioned immobilizing layer comprises an electrolytic polymer material which causes an oxidation reduction reaction with change in the hydrogen ion concentration.

Moreover, the present invention is a method of the detection of a nucleic acid having a specified base sequence in which the pyrophosphate detection sensor described above is used, the method comprising the steps of: (a) preparing a sample solution comprising a sample, a primer having a base sequence including a complementary binding region which complementarily binds to the aforementioned nucleic acid, and a nucleotide; (b) subjecting the aforementioned sample solution to a condition which causes an extension reaction of the aforementioned primer, and producing pyrophosphate when the aforementioned extension reaction is caused; (c) making a state in which the aforementioned sample solution is received in the aforementioned sample solution receptive part of the aforementioned pyrophosphate detection sensor; (d) measuring an electrochemical alteration with the change in hydrogen ion concentration of the aforementioned immobilizing layer by the aforementioned measurement means of the aforementioned pyrophosphate detection sensor; (e) detecting the aforementioned extension reaction on the basis of the measurement result in the step (d); and (f) detecting the aforementioned nucleic acid on the basis of the detection result in the step (e).

Furthermore, the present invention is a method of the discrimination of a base type in a base sequence of a nucleic acid in which the pyrophosphate detection sensor described above is used, the method comprising the steps of: (a) preparing a sample solution comprising a nucleic acid, a primer having a base sequence including a complementary binding region which complementarily binds to the aforementioned nucleic acid, and a nucleotide; (b) subjecting the aforementioned sample solution to a condition which causes an extension reaction of the aforementioned primer, and producing pyrophosphate when the aforementioned extension reaction is caused; (c) making a state in which the aforementioned sample solution is received in the aforementioned sample solution receptive part of the aforementioned pyrophosphate detection sensor; (d) measuring an electrochemical alteration with the change in hydrogen ion concentration of the aforementioned immobilizing layer by the aforementioned measurement means of the aforementioned pyrophosphate detection sensor; (e) detecting the aforementioned extension reaction on the basis of the measurement result in the step (d); and (f) discriminating the base type in the base sequence of the aforementioned nucleic acid on the basis of the detection result in the step (e).

Foregoing objects, other objects, aspects, and advantages of the present invention will be apparent from the following detailed description of the preferred embodiment with reference to the accompanying drawings.

### BRIEF DESCRIPTION OF THE DRAWINGS

FIG. 1 is a drawing schematically illustrating H⁺-pyrophosphatase in the state intrinsically included in a plant tonoplast membrane.

FIG. 2 is a cross sectional view schematically illustrating the constitution of a pyrophosphate detection sensor in first embodiment.

FIG. 3 is a cross sectional view schematically illustrating the constitution of a pyrophosphate detection sensor in second embodiment.

FIG. 4 is a cross sectional view schematically illustrating the constitution of a pyrophosphate detection sensor in third embodiment.

FIG. 5 is a cross sectional view schematically illustrating the constitution of a pyrophosphate detection sensor in fourth embodiment.

FIG. 6 is a cross sectional view schematically illustrating the constitution of a pyrophosphate detection sensor in fifth embodiment.

FIG. 7 is a cross sectional view schematically illustrating Constitution Example 1 of the pyrophosphate detection sensor.

FIG. 8 is a cross sectional view schematically illustrating Constitution Example 2 of the pyrophosphate detection sensor.

FIG. 9 is a cross sectional view schematically illustrating Constitution Example 3 of the pyrophosphate detection sensor.

FIG. 10 is a cross sectional view schematically illustrating Constitution Example 4 of the pyrophosphate detection sensor.

FIG. 11 is a drawing illustrating a reaction system when SNP sites are identical between a target DNA to be detected and a DNA probe.

FIG. 12 is a drawing illustrating a reaction system when SNP sites are not identical between a target DNA to be detected and a DNA probe.

FIG. 13A is a drawing illustrating two kinds of primer C and primer D which can completely hybridize to a specified base sequence of λDNA; FIG. 13B presents a Table showing compositions of PCR reaction mixtures G and H; and FIG. 13C is a flow chart showing reaction temperature condition in the PCR reaction performed.

FIG. 14A is a drawing illustrating wild type λDNA, mutated λDNA and a typing primer; FIG. 14B presents a Table showing compositions of PCR reaction mixtures I and J; and FIG. 14C is a flow chart showing reaction temperature condition in the PCR reaction performed.

### BEST MODE FOR CARRYING OUT THE INVENTION

Hereinafter, embodiments of the present invention are explained with reference to drawings.

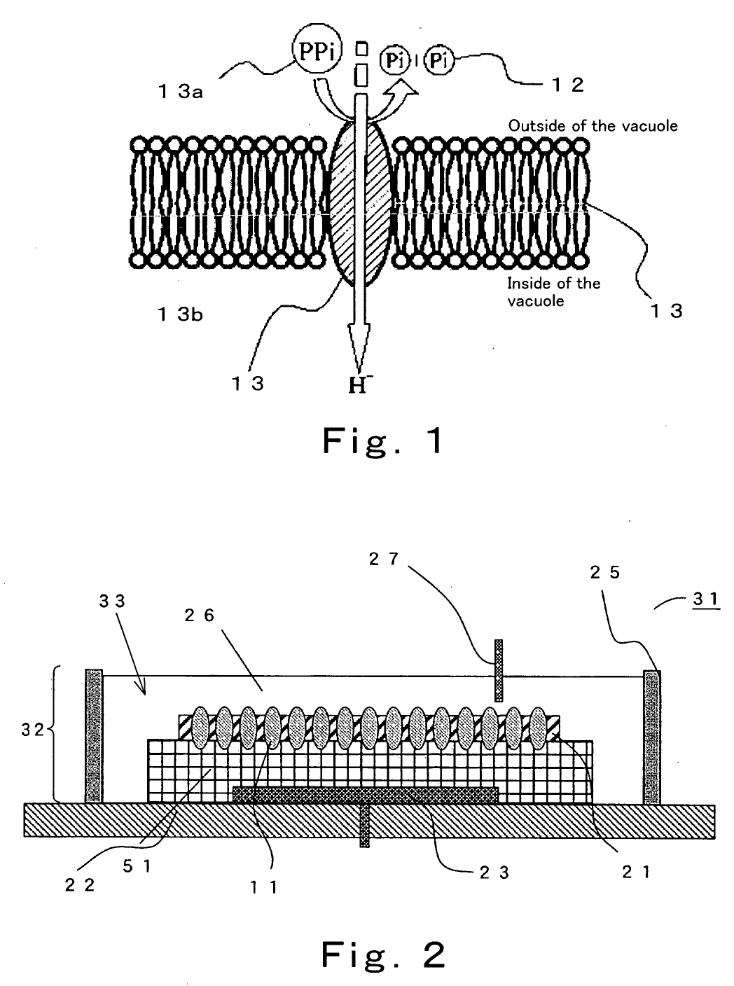

First, the reaction principle of the present invention is explained with reference to FIG. 1. In the present invention, H⁺-pyrophosphatase is used for qualitatively or quantitatively detecting pyrophosphate. FIG. 1 is a drawing schematically illustrating H⁺-pyrophosphatase in the state intrinsically included in a plant tonoplast membrane. As is shown in FIG. 1, H⁺-pyrophosphatase 11 is a membrane protein which is usually present within a tonoplast membrane 13 of plants and the like, and has a property to transport a hydrogen ion from the outside of the tonoplast membrane 13 (front face 13a side), which does not permeate or hardly permeates a hydrogen ion, toward inside of the tonoplast membrane 13 (back face 13b side) with the hydrolysis reaction which produced 2 molecules of phosphoric acid 12 from one molecule of pyrophosphate 10. Thus, the enzymatic reaction of the H⁺-pyrophosphatase increases the hydrogen ion concentration inside of the tonoplast membrane 13, and reduces the hydrogen ion concentration outside of the tonoplast membrane 13.

The pyrophosphate detection sensor according to each embodiment detects the pyrophosphate utilizing the property of H⁺-pyrophosphatase as described above and the mode being a membrane protein. More specifically, a region is separated by a membrane holding the H⁺-pyrophosphatase, and the change in at least either one of hydrogen ion concentration is measured, thereby enabling detection of the amount of the pyrophosphate, hydrolysis of which the H⁺-pyrophosphatase participated. Thus, the pyrophosphate detection sensor according to each embodiment detects pyrophosphate by detecting change in the hydrogen ion concentration which directly participated in the action of H⁺-pyrophosphatase, therefore, the detection in a convenient manner with high sensitivity is enabled. Also, for carrying out the detection as described above, separation of the region from which the hydrogen ion is transported and the region to which the hydrogen ion is transported becomes an essential requirement, however, the mode of the H⁺-pyrophosphatase can be utilized for the separation of the region on behalf of being a membrane protein. This is responsible for simplification of the constitution of the pyrophosphate detection sensor.

In the detection step using the pyrophosphate detection sensor according to each embodiment, the sample solution containing pyrophosphate is brought into contact with H⁺-pyrophosphatase in the state intrinsically included in a H⁺ impermeable membrane such as tonoplast membrane 13 isolated from a plant cell or the like.

Thereafter, change in the hydrogen ion concentration inside of the H⁺ impermeable membrane or outside of the H⁺ impermeable membrane is measured. Accordingly, presence or amount of the pyrophosphate in the sample solution is measured to execute qualitative detection or quantitative detection of pyrophosphate.

### First Embodiment

This embodiment concerns a pyrophosphate detection sensor. FIG. 2 is a cross sectional view schematically illustrating the constitution of the pyrophosphate detection sensor of this embodiment. The pyrophosphate detection sensor 31 comprises an insulation board 22, a solution holding part 32 formed with a solution holding member 25 immobilized on the insulation board 22, and a measurement means. The solution holding part 32 comprises an immobilizing layer 51 formed on the board 22, a H⁺ impermeable membrane 21 immobilized on the aforementioned immobilizing layer 51, and a sample solution receptive part 33 corresponding to a region not having these components constituted. The measurement means has a hydrogen ion sensitive electrode 23 provided just above the board 22 to be brought into contact with the immobilizing layer, and a reference electrode 27 provided to be brought into contact with a sample solution 26 in the state of the sample solution 26 being filled in the sample solution receptive part 33.

The H⁺ impermeable membrane 21 has H⁺-pyrophosphatase 11. The H⁺ impermeable membrane 21 comprises a membrane that is hardly hydrogen ion permeable except for the part with the H⁺-pyrophosphatase 11, and for example, a natural tonoplast membrane, an artificial lipid bilayer or the like can be utilized. In the H⁺ impermeable membrane 21, active site of the H⁺-pyrophosphatase 11 which hydrolyzes the pyrophosphate is exposed to the side of the sample solution receptive part 33.

The immobilizing layer 51 is formed with a material that can sufficiently permeate a hydrogen ion and can hold moisture. Further, the immobilizing layer 51 is formed with a material which can immobilize the H⁺ impermeable membrane 21 on the upper face thereof. The immobilizing layer 51 can be formed with a gel which immobilizes the H⁺ impermeable membrane 21 on the upper face thereof utilizing its holding capacity, or formed with a SAM membrane which immobilizes the H⁺ impermeable membrane 21 on the upper face thereof utilizing a crosslinking reaction. Examples of such materials which may be used include polymer gels such as agarose gels, materials comprising a fullerene-like compound, and the like.

The hydrogen ion sensitive electrode 23 may be anyone which can function as a general pH sensor, and examples thereof which can be utilized include glass electrodes, ISFET electrodes (ion sensitive FET; FET which is sensitive to an ion, in which an ion sensitive membrane is used for the gate), LAPS (Light-Addressable Potentiometric Sensor) and the like.

As the reference electrode 27, a standard hydrogen electrode, a silver/silver chloride electrode, a saturated calomel electrode or the like can be utilized.

The insulation board 22 and the solution holding part material 25 may be formed with any material that does not affect the hydrolyzing reaction of pyrophosphate, which can be e.g., glass, silicon, plastic and the like.

Method of the detection of pyrophosphate included in the sample solution 26 using the pyrophosphate detection sensor 31, and principle thereof are now explained. First, the sample solution 26 is filled in the sample solution receptive part 33. When pyrophosphate is included in the sample solution 26, the pyrophosphate is hydrolyzed into phosphoric acid by the activity of H⁺-pyrophosphatase 11, and along with this event, the hydrogen ion concentration in the immobilizing layer 51 is elevated. By measuring the elevated concentration using the hydrogen ion sensitive electrode 23, the pyrophosphate concentration in the sample solution 26 for the measurement can be detected. Specifically, by measuring change in the electric potential difference between the hydrogen ion sensitive electrode 23 and the reference electrode 27, the hydrogen ion concentration in the immobilizing layer 51 is measured, thereby detecting the pyrophosphate concentration in the sample solution 26 for the measurement based on the result of the measurement.

The H⁺ impermeable membrane 21 may include the H⁺-pyrophosphatase whose active site that hydrolyzes the pyrophosphate is exposed to the side of the immobilizing layer 51 (inside). However, when the H⁺ impermeable membrane 21 having the H⁺-pyrophosphatase whose active site that hydrolyzes the pyrophosphate is exposed to inside is used, concentration of the pyrophosphate of the immobilizing layer 51 is preferably set to be lower than the concentration of pyrophosphate in the sample solution 26, and most preferably, the immobilizing layer 51 does not contain pyrophosphate. Accordingly, transport of the hydrogen ion from the immobilizing layer 51 to the sample solution 26 is diminished or avoided, resulting in dominant transport of the hydrogen ion from the sample solution 26 to the immobilizing layer 51. Thus, change in the hydrogen ion concentration of the immobilizing layer 51 is approximately limited to that resulting from the pyrophosphate included in the sample solution 26. Therefore, the amount of the pyrophosphate included in the sample solution 26 can be accurately estimated.

Because the pyrophosphate detection sensor 31 is not constituted such that entire boundary face of the immobilizing layer 51 with the sample solution receptive part 33 is not covered by the H⁺ impermeable membrane 21, the hydrogen ion can migrate between the sample solution 26 and the immobilizing layer 51 via the boundary part not covered by the H⁺ impermeable membrane 21. Although the hydrogen ion is diffused from this part to keep the equilibrium, migration of the hydrogen ion by way of the diffusion is slower compared to migration of the hydrogen ion by way of the H⁺-pyrophosphatase 11 activity. Therefore, the change in the hydrogen ion concentration as measured by the hydrogen ion sensitive electrode 23 can be concluded to approximately result from the activity of the H⁺-pyrophosphatase 11.

### Second Embodiment

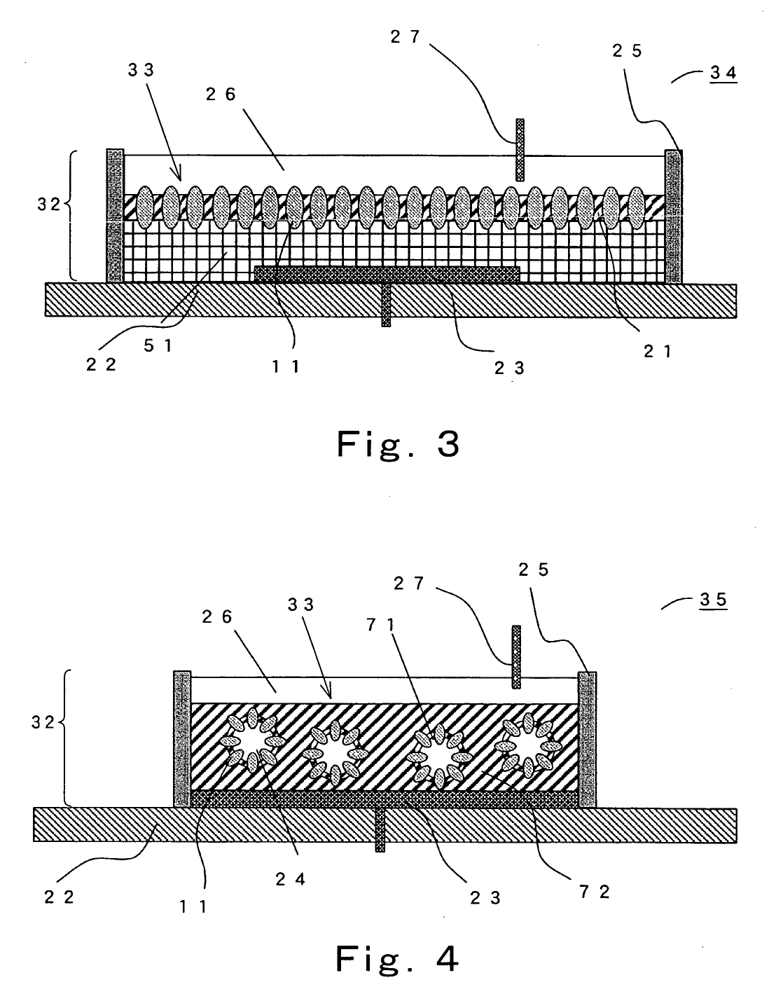

This embodiment concerns a pyrophosphate detection sensor. FIG. 3 is a cross sectional view schematically illustrating the constitution of the pyrophosphate detection sensor of this embodiment. The pyrophosphate detection sensor 34 of this embodiment is different from the pyrophosphate detection sensor 31 of the first embodiment only in terms of the immobilizing layer 51 being formed on the entire region just above the insulation board 22, and the H⁺ impermeable membrane 21 being provided over the entire boundary region between the immobilizing layer 51 and the sample solution receptive part 33. Because other constitution is identical to that in the pyrophosphate detection sensor 31 of the first embodiment, the explanation thereof is omitted.

### Third Embodiment

This embodiment concerns a pyrophosphate detection sensor. FIG. 4 is a cross sectional view schematically illustrating the constitution of the pyrophosphate detection sensor of this embodiment. Differences from the first embodiment exist in the H⁺ impermeable membrane being formed with membrane vesicles 71, and in the position where the H⁺ impermeable membrane, which is a membrane vesicle 71, is immobilized. Hereinafter, only the differences from the first embodiment are explained.

The membrane vesicles 71 have the H⁺-pyrophosphatase 11, and are immobilized within the immobilizing layer 51. The immobilizing layer 51 comprises, for example, a material which can immobilize the membrane vesicles 71 inside thereof by means of the holding capacity of a gel. The membrane vesicle 71 for use may be any one which is prepared from the vacuole isolated from a cell. Also, the membrane vesicle 71 for use may be any one formed by reconstituting a membrane which does not permeate or hardly permeates a hydrogen ion, such as an artificially formed lipid bilayer membrane or an LB membrane, to intrinsically include isolated and purified H⁺-pyrophosphatase therein.

The membrane of the membrane vesicle 71 may include a protein other than the H⁺-pyrophosphatase. However, such a protein is preferably a protein that does not react with pyrophosphate, or that has low reactivity therewith. More specifically, when the pyrophosphate reacts with the protein other than the H⁺-pyrophosphatase being present in the membrane of the membrane vesicle 71, the amount of the pyrophosphate that reacts with the H⁺-pyrophosphatase is decreased, thereby leading to decrease in the amount of transport of H⁺. In addition, when a protein that does not react with the pyrophosphate and transports a hydrogen ion through a reaction with a substance other than the pyrophosphate is included in the membrane of the membrane vesicle 71, it is preferred that the sample solution 26 scarcely contains a substance that reacts with the protein. Specifically, for example, when the membrane of the membrane vesicle 71 contains ATPase which is a protein that hardly reacts with the pyrophosphate and transports the hydrogen ion through the reaction with ATP, it is preferred that the sample solution 26 scarcely contains ATP.

Method of the detection of pyrophosphate included in the sample solution 26 using the pyrophosphate detection sensor 35, and principle thereof are now explained. First, the sample solution 26 is filled in the sample solution receptive part 33. When pyrophosphate is present in the sample solution 26, the pyrophosphate is diffused into the immobilizing layer 51. Then, the pyrophosphate diffused in the immobilizing layer 51 is hydrolyzed by the activity of the H⁺-pyrophosphatase 11 to give phosphoric acid, accompanied by elevation of the hydrogen ion concentration in the internal liquid 24 of the membrane vesicles 71, while accompanied by decrease in the hydrogen ion concentration around the H⁺-pyrophosphatase 11. When the H⁺-pyrophosphatase is present quite close to the hydrogen ion sensitive electrode 23, decrease in the hydrogen ion concentration is measured by the hydrogen ion sensitive electrode 23, thereby enabling the measurement of pyrophosphate concentration in the sample solution 26.

### Fourth Embodiment

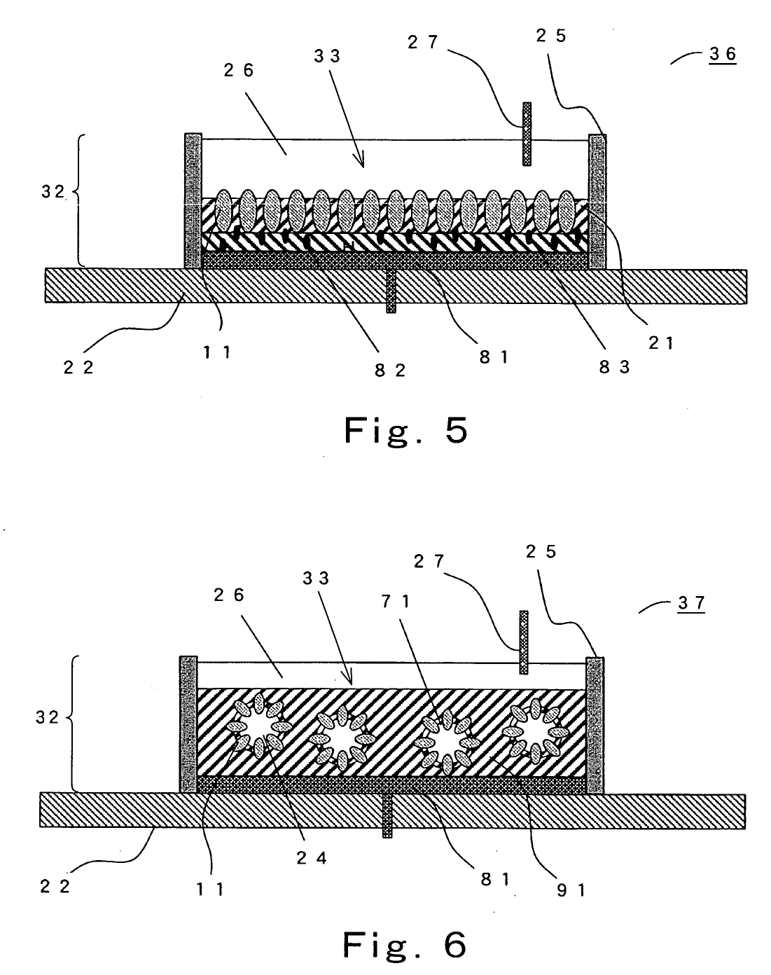

This embodiment concerns a pyrophosphate detection sensor. FIG. 5 is a cross sectional view schematically illustrating the constitution of the pyrophosphate detection sensor of this embodiment. The pyrophosphate detection sensor 36 is different from the sensor of the second embodiment only in terms of the constitution of the immobilizing layer and the constitution of the measurement electrode. Hereinafter, explanation is made in this respect.

The measurement electrode consists of a polarizable electrode 81 formed on the insulation board 22. The polarizable electrode 81 can be constituted from an electrode which can be used in conventional electrochemical measurement such as gold, platinum, carbon or the like. For the polarizable electrode 81, an electrode having an extremely simple constitution can be utilized. This is responsible for simplification of the overall constitution of the pyrophosphate detection sensor. Furthermore, according to this embodiment, in addition to a standard hydrogen electrode, silver/silver chloride electrode, saturated calomel electrode and the like, an electrode such as gold, platinum, carbon or the like can be utilized as the reference electrode 27, which can additionally contribute to simplification of the overall constitution of the pyrophosphate detection sensor.

An immobilizing layer 83 containing a mediator 82 is formed on the surface of the polarizable electrode 81. For the immobilizing layer 83, for example, a SAM (self-assembled monolayer) membrane can be used in which a straight-chain carbon compound having a thiol group at one end is utilized. However, the immobilizing layer 83 is not limited thereto as long as it is formed with a material which can immobilize the H⁺ impermeable membrane 21, but may be formed with a gel which immobilizes the H⁺ impermeable membrane 21 by its holding capacity. The mediator 82 which may be used is an oxidized product of a hydrogen ion sensitive substance. On thus formed immobilizing layer 83 is immobilized the H⁺ impermeable membrane 21 containing H⁺-pyrophosphatase. When the SAM membrane as described above is used for the immobilizing layer 83, the H⁺ impermeable membrane 21 can be immobilized on the upper face of the immobilizing layer 83 by a crosslinking reaction of the thiol group. When the H⁺ impermeable membrane 21 is a lipid membrane, hydrophobic parts of the lipid membrane and the immobilizing layer 83 are opposed. Thus, the hydrophilic part of the lipid membrane forms a membrane surface. Although the H⁺-pyrophosphatase 11 is immobilized inside of the membrane which is formed with the hydrophobic part of the lipid membrane and the immobilizing layer 83, the active site that hydrolyzes the pyrophosphate of H⁺-pyrophosphatase 11 then is exposed to outside of the H⁺ impermeable membrane 21.

Method of the detection of pyrophosphate included in the sample solution 26 using the pyrophosphate detection sensor 36, and principle thereof are now explained. First, the sample solution 26 is filled in the sample solution receptive part 33. When pyrophosphate is present in the sample solution 26, the pyrophosphate is hydrolyzed into phosphoric acid by the activity of H⁺-pyrophosphatase 11, and along with this event, the hydrogen ion concentration in the immobilizing layer 83 is elevated. When the oxidized product of a hydrogen ion sensitive mediator 82 is present then, a reduced product of the mediator 82 is produced by an oxidation reduction reaction. By applying to the polarizable electrode 81 sufficiently higher electric potential than the oxidation reduction potential of the mediator 82, electric current corresponding to the concentration of the reduced substance of the mediator 82 can be measured. Therefore, detection of the concentration of the pyrophosphate in the sample solution 26 is enabled.

### Fifth Embodiment

This embodiment concerns a pyrophosphate detection sensor. FIG. 6 is a cross sectional view schematically illustrating the constitution of the pyrophosphate detection sensor of this embodiment. The pyrophosphate detection sensor 37 of this embodiment is different from the sensor of the fourth embodiment only in terms of the H⁺ impermeable membrane being formed with membrane vesicles 71, position where the H⁺ impermeable membrane that is the membrane vesicle 71 is immobilized, and the constitution of the immobilizing layer. Hereinafter, explanation is made with respect to only the difference from the fourth embodiment.

The membrane vesicle 71 has the H⁺-pyrophosphatase 11, and is immobilized within an immobilizing layer 91. The immobilizing layer 91 consists of an electrolytic polymerized membrane. In the process for immobilizing the membrane vesicles with an electrolytic polymerized membrane, for example, a monomer prior to polymerization and the membrane vesicles 71 are mixed, and the formation can be achieved by applying a predetermined electric potential. As the electrolytic polymer material for forming the electrolytic polymerized membrane, any one which is electrochemically active can be selected, and for example, poly(aniline), poly(o-phenylenediamine), poly(N-methylaniline), poly(pyrrol), poly(N-methylpyrrol), poly(thiophene) or the like can be used.

Method of the detection of pyrophosphate included in the sample solution 26 using the pyrophosphate detection sensor 37, and principle thereof are now explained. First, the sample solution 26 is filled in the sample solution receptive part 33. When pyrophosphate is present in the sample solution 26, the pyrophosphate is diffused into the immobilizing layer 91. Then, the pyrophosphate is hydrolyzed by the activity of the H⁺-pyrophosphatase 11 to give phosphoric acid. Upon hydrolysis of the pyrophosphate, the hydrogen ion concentration in the internal liquid 24 is elevated, whereas the hydrogen ion concentration is decreased around the H⁺-pyrophosphatase 11. This change in the hydrogen ion concentration causes an oxidation reduction reaction of the electrolytic polymerized membrane that is the immobilizing layer 91, whereby enabling detection of the pyrophosphate concentration in the sample solution 26 through measuring the electron migration with the polarizable electrode 81.

In the pyrophosphate detection sensor according to the present invention, as is clear from the reaction principle explained with reference to FIG. 1, a field where a hydrogen ion or an oxidized product of a hydrogen ion sensitive mediator can be present in the ionic state is required between the H⁺ impermeable membrane containing the H⁺-pyrophosphatase and the measurement electrode (hydrogen ion sensitive electrode 23, polarizable electrode 81). It is also possible to employ an aqueous bulk solution as such a field. However, for allowing the aqueous bulk solution to be present between the H⁺ impermeable membrane and the measurement electrode, it is necessary to manufacture a sensor through extremely complicated steps as suggested in, for example, JP-A-H6-90736. Furthermore, thus manufactured sensor can be stored only in an aqueous solution once manufactured. Thus, when the pyrophosphate detection sensor is used, for example, as a DNA detection sensor, the handling method and storing method thereof may be significantly specific. Therefore, taking into account also of the complexity in the manufacture method, it is not suited for e.g., use in laboratory tests in disposable format and the like. On the other hand, in the pyrophosphate detection sensor according to the first to fifth embodiments, the aforementioned field where a hydrogen ion or an oxidized product of the hydrogen ion sensitive mediator can be present in the ionic state consists of an immobilizing layer. Such an immobilizing layer can be formed with, for example, a SAM membrane or an electrolytic polymerized membrane. Accordingly, the sensor can be manufactured by a comparatively simple method. Also, the sensor having an immobilizing layer consisting of a polymer gel can be stored with water molecules being held, therefore, handling and storage may be extremely simplified. Also in cases where the immobilizing layer is formed with other material, handling and storage may be so simplified in comparison with the cases in which the aforementioned field is constituted with the aqueous bulk solution. Therefore, constitution suited for use in also, e.g., laboratory tests in disposable format is permitted. Additionally, by decreasing the thickness of the immobilizing layer as small as possible, rate of change in the hydrogen ion concentration can be elevated, thereby enabling the improvement of the sensitivity.

Hereinafter, other Constitution Examples of the pyrophosphate detection sensor in which H⁺-pyrophosphatase is used are illustrated.

<Constitution Example 1 of Pyrophosphate Detection Sensor>

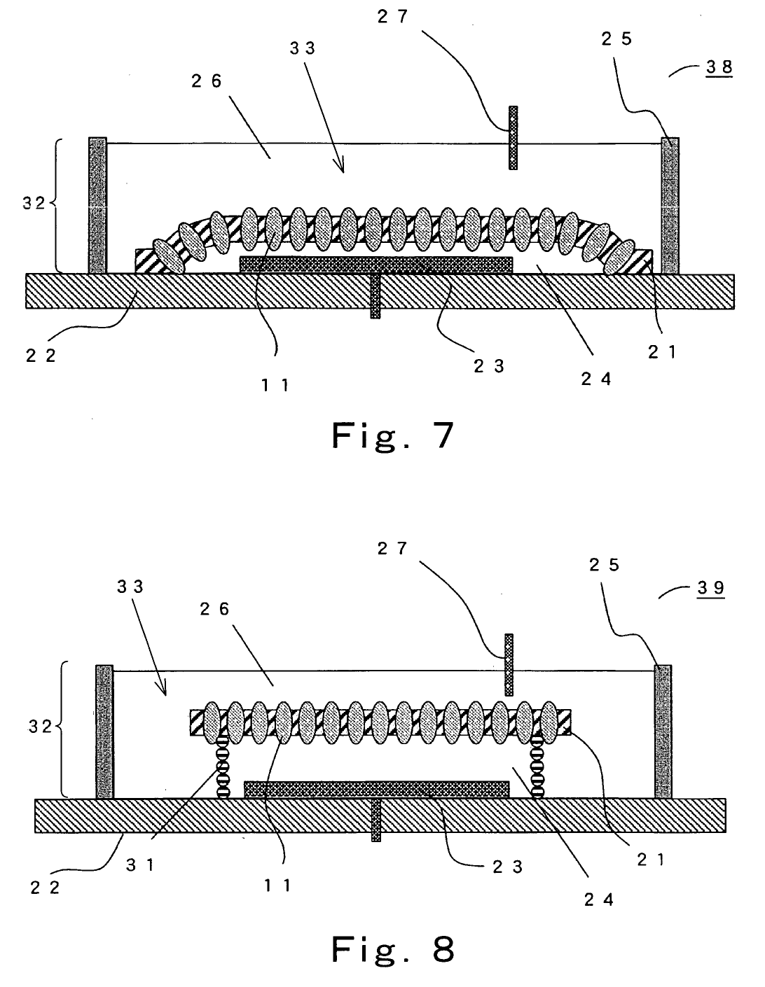

FIG. 7 is a cross sectional view schematically illustrating one Constitution Example of the pyrophosphate detection sensor. It is different from the sensor in the first embodiment only in terms of the periphery of the hydrogen ion sensitive electrode 23 being filled with the internal liquid 24, and the H⁺ impermeable membrane 21 being immobilized on the insulation board 22 such that it covers the hydrogen ion sensitive electrode 23. Hereinafter, explanation is made with respect to only the difference from the first embodiment.

Process for immobilizing the H⁺ impermeable membrane 21 may be any process as long as the H⁺ impermeable membrane 21 covers the entire surface of the hydrogen ion sensitive electrode 23, and for example, a process of transferring to a SAM membrane utilizing liposome, or an LB process may be used. Of the region in the solution holding part 32 which is separated by the H⁺ impermeable membrane 21, the region including the hydrogen ion sensitive electrode 23 should be filled with an internal liquid 24.

Method of the detection of pyrophosphate included in the sample solution 26 using the pyrophosphate detection sensor 38, and principle thereof are now explained. First, the sample solution 26 is filled in the sample solution receptive part 33. When pyrophosphate is included in the sample solution 26, the pyrophosphate is hydrolyzed into phosphoric acid by the activity of H⁺-pyrophosphatase 11, and along with this event, the hydrogen ion concentration in the internal liquid 24 is elevated. By measuring the elevated concentration using the hydrogen ion sensitive electrode 23, the pyrophosphate concentration in the sample solution 26 for the measurement can be detected.

The internal liquid 24 is not particularly limited, however, when the H⁺-pyrophosphatase is included whose active site for the pyrophosphate is exposed to the region of the side of the hydrogen ion sensitive electrode 23 (inside) in the H⁺ impermeable membrane 21, the concentration of pyrophosphate in the internal liquid 24 is preferably set to be lower than the concentration of the pyrophosphate in the sample solution 26, and most preferably, the internal liquid 24 does not contain pyrophosphate. Accordingly, transport of the hydrogen ion from the internal liquid 24 to the sample solution 26 is diminished or avoided, resulting in dominant transport of the hydrogen ion from the sample solution 26 to the internal liquid 24. Thus, change in the hydrogen ion concentration of the internal liquid 24 is approximately limited to that resulting from the pyrophosphate included in the sample solution 26. Therefore, the amount of the pyrophosphate included in the sample solution 26 can be accurately estimated.

<Constitution Example 2 of Pyrophosphate Detection Sensor>

FIG. 8 is a cross sectional view schematically illustrating one Constitution Example of the pyrophosphate detection sensor. It is different from the sensor in the Constitution Example 1 described above only in terms of the process for immobilizing the H⁺ impermeable membrane 21. Hereinafter, explanation is made with respect to only the difference from the Constitution Example 1.

The H⁺ impermeable membrane 21 of the pyrophosphate detection sensor 39 according to this Constitution Example is immobilized on the insulation board 22 via a straight-chain carbon compound 31.

<Constitution Example 3 of Pyrophosphate Detection Sensor>

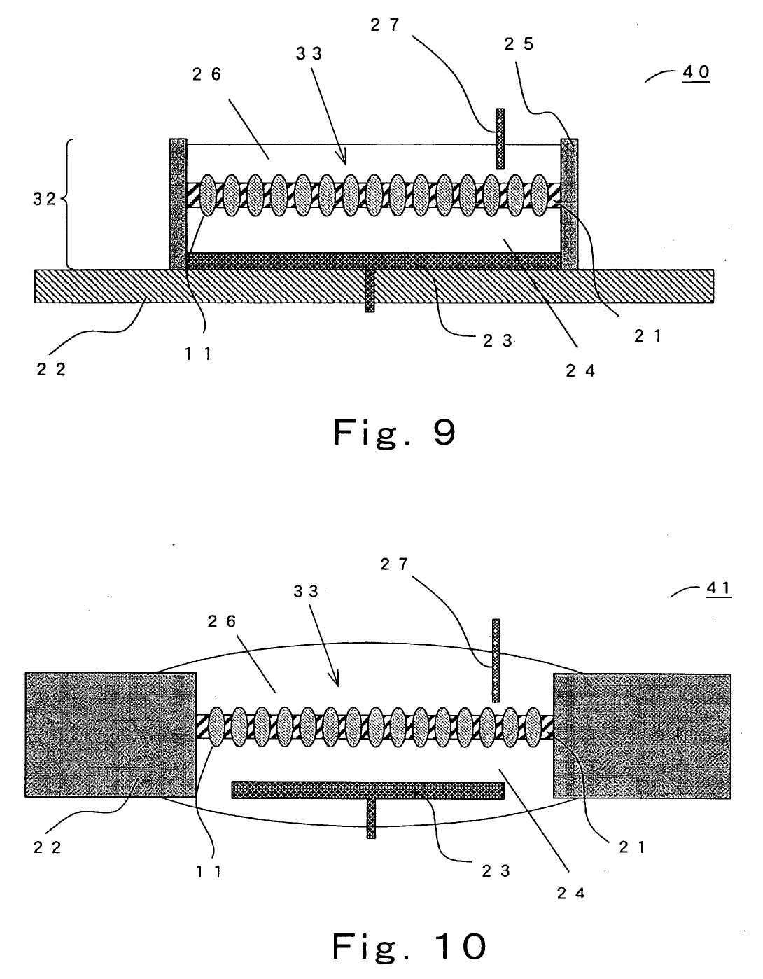

FIG. 9 is a cross sectional view schematically illustrating one Constitution Example of the pyrophosphate detection sensor. It is different from the sensor in the Constitution Example 1 described above only in terms of the process for immobilizing the H⁺ impermeable membrane 21. Hereinafter, explanation is made with respect to only the difference from the Constitution Example 1.

The H⁺ impermeable membrane 21 of the pyrophosphate detection sensor 40 according to this Constitution Example is immobilized at both ends to the solution holding part material 25.

<Constitution Example 4 of Pyrophosphate Detection Sensor>

FIG. 10 is a cross sectional view schematically illustrating one Constitution Example of the pyrophosphate detection sensor. In the pyrophosphate detection sensor 41 according to this Constitution Example, the H⁺ impermeable membrane 21 is immobilized in a through-hole formed in the insulation board 22. The hydrogen ion sensitive electrode 23 is provided inside of the H⁺ impermeable membrane 21, while the reference electrode 27 is provided outside thereof. The hydrogen ion sensitive electrode 23 and the reference electrode 27 may be formed on the insulation board 22. The internal liquid 24 and the sample solution 26 are in contact with the hydrogen ion sensitive electrode 23 and the reference electrode 27, respectively, and can be in contact with the H⁺ impermeable membrane 21. Orientation of the H⁺-pyrophosphatase is similar to that in the first embodiment. Process for immobilizing the H⁺ impermeable membrane 21 in the through-hole can be executed according to a known process in which, e.g., Langmuir-Blodgette method is utilized (see, Books-Yoshioka, edited by Yasunobu Okada, “Novel patch clamp experimental techniques (Shin Patch clamp zikken gizyutu-hou)” p. 214). In addition, method of the formation of the through-hole and well on the insulation board, and formation of the electrode on the insulation board 22 may be carried out according to, for example, etching of a silicon board or the like (see, JP-A-2004-12215).

Method of the detection of pyrophosphate in the sample solution 26, and principle thereof are similar to that in Constitution Example 1, therefore, the explanation is omitted.

### Sixth Embodiment

Sixth embodiment concerns a method of the detection of a DNA having a specified sequence using the pyrophosphate sensor according to the present invention.

In this embodiment, a sample is first subjected to a reaction system including a DNA probe having a sequence complementary to the sequence of a target DNA, DNA polymerase and deoxynucleotide. The “reaction system” herein refers to a series of nucleic acid extension reactions as explained below, and a field where such reactions are executed. In the “reaction system”, there exist components required for executing the series of the reactions. The “reaction system” can be usually provided in the form of a solution including the components described above dissolved in a suitable solvent (for example, Tris-HCl buffer, any buffer which can be generally used in a nucleic acid extension reaction or a nucleic acid amplification reaction (including buffers in commercially available kits)). The DNA polymerase may be any arbitrary DNA polymerase which is commercially available, or can be prepared by a person skilled in the art. Preferably, Taq polymerase can be used, but not limited thereto. The deoxynucleotide may be each deoxynucleoside triphosphate (also referred to as dNTP: including deoxycytosine triphosphate, deoxyguanine triphosphate, deoxyadenine triphosphate, and deoxythymidine triphosphate), and is a substance which can be generally used as a direct precursor of DNA synthesis. Accordingly, the DNA probe is extended, whereby leading to production of pyrophosphate with the extension reaction of the DNA probe. In connection with this reaction, explanation is made with reference to the chemical reaction formula 1.

When this DNA probe if hybridized to the target DNA, extension is performed through incorporating one deoxynucleotide (in the chemical reaction formula 1; dNTP) in the reaction system by the DNA polymerase that is present in the reaction system to produce one molecule of pyrophosphate.

[Chemical reaction formula 1]
DNA₍ₙ₎+dNTP⇄DNA₍ₙ₊₁₎+PPi

In the chemical reaction formula 1, the subscript “n+1” indicates that the DNA probe having “n” bases is extended to have “n+1” bases. By detecting the pyrophosphate, which was produced in this manner, using the pyrophosphate detection sensor according to each embodiment described above, detection of the DNA having a specified sequence in a sample solution is enabled.

Specifically, a DNA having a specified sequence can be detected through the steps of: (c) filling the aforementioned sample solution containing pyrophosphate in the sample solution receptive part 33 of any one of the pyrophosphate detection sensors described above; (d) electrochemically measuring change in the hydrogen ion concentration of the immobilizing layer by the measurement means of the pyrophosphate detection sensor; (e) detecting the extension reaction of the DNA on the basis of the measurement result in the step (d); and (f) detecting the aforementioned DNA on the basis of the detection result in the step (e).

The DNA probe for use in this embodiment is designed such that it has a sequence complementary to the sequence of a target DNA to be detected. This DNA probe serves as a primer for DNA probe extension when it is hybridized to the sequence of a target DNA to be detected. Accordingly, the length of the DNA probe is sufficiently long to serve as a primer for an extension reaction. For example, it can have a length of at least 10 bases, at least 12 bases, at least 15 bases, at least 20 bases, and at least 30 bases. In light of the possibility of carrying out satisfactory hybridization and primer extension, and the ease of preparation thereof, the length of 20 to 25 bases is preferred. The DNA probe for use in the method of the present invention may have any length as long as it specifically hybridizes to the target DNA to be detected, and serves as a primer for extension of the DNA probe.

When the sequence to be detected is known, the DNA probe can be designed such that it becomes completely complementary to this sequence, i.e., that it has a sequence which exactly corresponds to bases in the sequence (A-T or C-G pair) in order to specifically hybridize to the target DNA in the sample solution, and to serve as a primer. When any target DNA having the specified sequence is not present in the sample solution, as a matter of course, hybridization to the DNA probe is not caused. Therefore, use of this reaction system enables detection of the presence of a sequence which is completely complementary to this DNA probe irrespective of the sequence to be detected being either known or unknown.

The hybridization and extension reaction can be performed under an arbitrary condition which allows DNA hybridization, and DNA extension reaction with the primer and deoxynucleotide by the action of the DNA polymerase to proceed. Hybridization of a DNA probe to the target DNA is carried out by a method described in experimental notes such as, for example, Sambrook et. al., (1989) Molecular Cloning: A Laboratory Manual, Second Edition, Vol. 1 to 3, Cold Spring Harbor Laboratory, and the like. This method has been known to persons skilled in the art.

Amount of the DNA probe, polymerase and deoxynucleotide which can be included in the reaction system may be determined ad libitum by a person skilled in the art.

Also, as illustrated in the chemical reaction formula 1, one molecule of the pyrophosphate is produced every time when one deoxynucleotide extension is executed. Therefore, when the lengths (bases) of both of the target DNA having the specified sequence and DNA probe are known, the target DNA having the specified sequence can be quantitatively detected.

In addition, by substituting the reaction represented by the chemical reaction formula 1 for an amplification reaction of a nucleic acid such as PCR, amount of the target DNA having the specified sequence in a sample can be significantly increased. Method of the PCR amplification has been known in this technical field (PCR Technology: Principles and Applications for DNA Amplification, edited by H A Erlich, Freeman Press, New York, N.Y. (1992); PCR Protocols: A Guide to Methods and Applications, edited by Innis, Gelf land, Snisky, and White, Academic Press, San Diego, Calif. (1990); Mattila et al., (1991) Nucleic Acids Res. 19: 4967; Eckert, K. A. and Kunkel, T. A. (1991) PCR Methods and Applications 1: 17; PCR, McPherson, Quirkes, and Taylor, IRL Press, Oxford). Use of the amplification reaction of a nucleic acid such as PCR also enables detection of a DNA in a trace amount.

### Seventh Embodiment

Seventh embodiment of the present invention is a method of the discrimination of a base type of a DNA in a measurement system, more specifically, a method of typing a SNP at a high speed, using the pyrophosphate detection sensor according to the present invention.

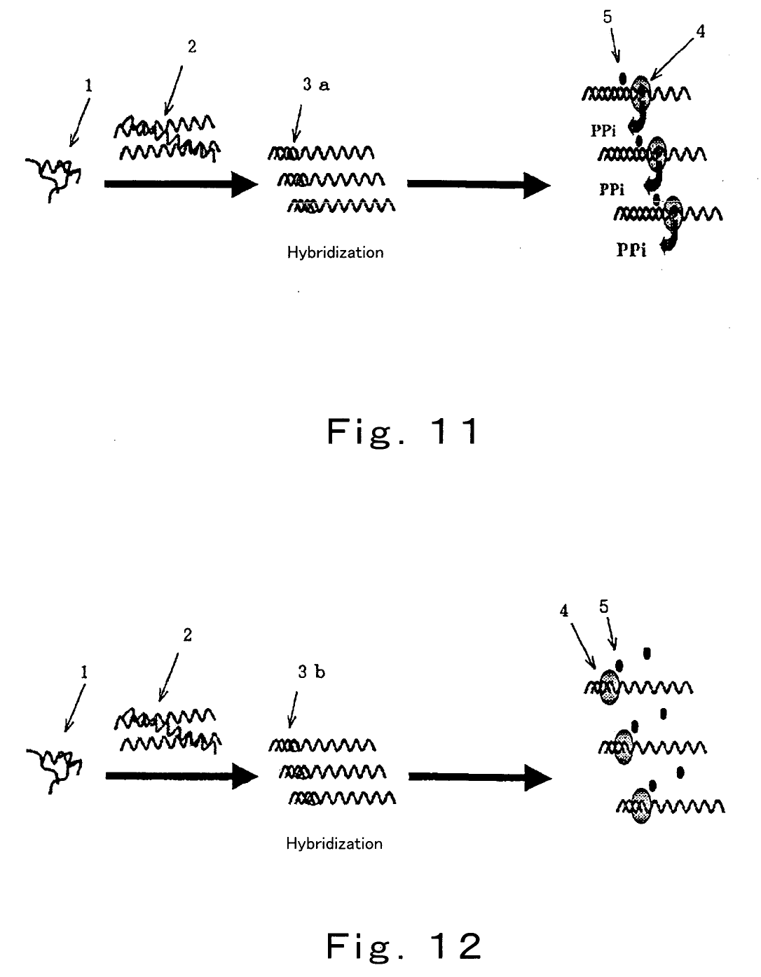

The seventh embodiment is explained with reference to FIG. 11 and FIG. 12. FIG. 11 shows a reaction system when SNP sites are identical between a target DNA to be detected and a DNA probe, while FIG. 12 shows a reaction system when SNP sites are not identical between a target DNA to be detected and a DNA probe. In FIG. 11 and FIG. 12, the numeral 1 denotes a DNA probe; the numeral 2 denotes a target DNA having a specified sequence; the symbol 3a denotes identical SNP sites; the symbol 3b denotes nonidentical SNP sites; the numeral 4 denotes DNA polymerase; and the numeral 5 denotes dNTP.

In this embodiment, a sample is first subjected to a reaction system including a DNA probe 1 having a sequence which is complementary to the target DNA sequence with the 3+ end being the SNP site, DNA polymerase 4, and deoxynucleotide 5. Thus, the DNA probe 1 is extended, whereby pyrophosphate produced with this extension reaction of the DNA probe 1.

The DNA probe 1 for use in this embodiment can be designed such that it is complementary to the target sequence, and that the 3′ end is the SNP site. In this embodiment, the DNA probe 1 may be designed similarly to that in the sixth embodiment described above except that the 3′ end is the SNP site. The DNA polymerase 4 and deoxynucleotide 5 for use in this embodiment may be similar to those in the sixth embodiment described above. Conditions of the hybridization and extension in this embodiment may be also similar to those in the sixth embodiment described above. The DNA probe in this embodiment may be any one as long as typing of the base type in a SNP site is enabled, but not limited to any probe designed as described above.

When base sequences of the target DNA 2 and the DNA probe 1 in a sample solution are completely complementary including the SNP site, the DNA probe 1 hybridizes to the target DNA 2, and serves as a primer for additional extension of the probe 1. In this instance, as shown in FIG. 11, one deoxynucleotide 5 extension is executed to the DNA probe 1 by the DNA polymerase 4 that is present in the reaction system thereby producing one molecule of pyrophosphate. On the other hand, when the base sequences of the target DNA 2 and the DNA probe 1 in the sample solution are not complementary at the SNP site (even though they are complementary in other part), the DNA probe 1 can hybridize to the target DNA 2, while it does not serve as a primer for the extension of the probe 1 due to mismatch at the 3′ end of the DNA probe 1. In this instance, as shown in FIG. 12, the reaction represented by the chemical formula 1 is not caused even though the DNA polymerase 4 and the necessary deoxynucleotide 5 are present in the reaction system. Therefore, pyrophosphate is not produced.

Accordingly, by detecting the pyrophosphate in the sample solution following the extension reaction of the DNA using the pyrophosphate detection sensor of each embodiment described above, the target DNA 2 having the specified sequence and the DNA probe 1 in the sample solution can be discriminated as being completely identical including the SNP site. When at most 4 kinds of probes 1 at the SNP site are used, typing of the SNP of the DNA 2 having the specified sequence in the sample on four types of bases is enabled.

Needless to add, four kinds of the probes are not necessarily required for the typing when the base type at the SNP site is known.

The method of typing a SNP by detecting the pyrophosphate in the sample solution following the DNA extension reaction using the pyrophosphate detection sensor of each embodiment described above comprises, more specifically, the steps of: (c) filling the aforementioned sample solution containing pyrophosphate in the sample solution receptive part 33 of any one of the pyrophosphate detection sensors described above; (d) electrochemically measuring change in the hydrogen ion concentration of the immobilizing layer by the measurement means of the pyrophosphate detection sensor; (e) detecting the extension reaction of the DNA on the basis of the measurement result in the step (d); and (f) discriminating the base type at the SNP site in the base sequence of the DNA on the basis of the detection result in the step (e).

The “sample solution” used in the present invention refers to any sample solution which may contain pyrophosphate. In the sixth and seventh embodiments, it is a sample solution which may contain a DNA from which pyrophosphate is produced by the extension reaction. Particularly, in the method relating to the detection of a DNA (sixth or seventh embodiment), the “sample solution” may be derived from an arbitrary analyte which may include the target DNA. When the target DNA may relate to a disease, such an analyte may be a cell, tissue, organ, or blood suffering from the disease. As a matter of course, the method of the present invention may be used in any fields but not limited to clinical applications. Therefore, such an analyte may be a cell, tissue, organ, or blood in which the target DNA is expressed or the presence is ascertained. The DNA may be extracted from such an analyte with a conventional method such as phenol extraction method and alcohol precipitation. Purity of the DNA may affect the efficiency of the reaction. Purification procedures of a DNA are also known to persons skilled in the art.

As explained in the foregoings, the present invention provides a pyrophosphate detection sensor which enables the measurement of pyrophosphate with high sensitivity, at a high speed, and in a quantitative manner; and a method of the detection of a nucleic acid and a method of the discrimination of a base type using the same.

Further, according to the present invention, presence of a target nucleic acid can be quantitatively measured without labeling the target DNA in a sample by measuring pyrophosphate produced with the extension reaction of the DNA. Still more, typing of the target SNP can be determined with high sensitivity and at a high speed.

### EXAMPLE 1

This Example relates to manufacture of the pyrophosphate detection sensor according to the fourth embodiment.

First, according to the method of Shizuo Yoshida et al., (Masayoshi Maeshima and Shizuo Yoshida, (1989) J. Biol. Chem. 264 (33), 20068-20073), membrane vesicles consisting of a tonoplast membrane 13 derived from Phaseolus aureus were placed in a solution consisting of a Tris/Mes buffer (concentration: 5 mM, pH 7.0), sorbitol (concentration: 0.25 M), DTT (concentration: 2 mM), to give a suspension of the membrane vesicles consisting of the tonoplast membrane 13 which includes H⁺-pyrophosphatase.

On the other hand, a gold electrode (polarizable electrode 81) was immersed in a 1 mM n-octanethiol/ethanol solution, and left to stand at room temperature for 4 hours to form a SAM membrane of octanethiol (immobilizing layer 83) on the surface of the gold electrode. Next, the octanethiol-modified electrode was immersed in a 10 mM aqueous thionine solution, and left to stand at room temperature for 1 hour to immobilize thionine (mediator 82) between the SAM membranes.

By adding the suspension of the membrane vesicles containing H⁺-pyrophosphatase dropwise to thus manufactured thionine/octanethiol-modified electrode, the H⁺-pyrophosphatase was immobilized on the surface of the immobilizing layer 83, thereby constituting the H⁺-pyrophosphatase electrode. A sodium pyrophosphate solution was brought into contact with the H⁺-pyrophosphatase such that each final concentration of sodium pyrophosphate became 20 μM, 40 μM, 60 μM, 80 μM and 100 μM, respectively, to initiate the hydrolyzing reaction of the pyrophosphate by the H⁺-pyrophosphatase.

A result was obtained showing approximately straight-line relationship between the concentration of sodium pyrophosphate in the sample liquid, and the electric current value indicated by the gold electrode when the electric potential of 200 mV was applied using a silver/silver chloride electrode as a reference electrode 27. Accordingly, it was proven that the amount of pyrophosphate can be measured by the present method.

### EXAMPLE 2

This Example relates to manufacture of the pyrophosphate detection sensor according to the first embodiment.

First, according to the method of Masasuke Yoshida et al., (Masa H. Sato, Masahiko Kasahara, Noriyuki Ishii, Haruo Homareda, Hideo Matsui and Masasuke Yoshida, (1994) J. Biol. Chem. 269 (9), 6725-6728), purification of vacuolarmembranous H⁺-pyrophosphatase was carried out from seeds of pumpkin.

On the other hand, a solution prepared by dissolving 0.1 g of polyvinylbutyral resin and 1 g of hexamethylenediamine in dichloromethane followed by stirring at room temperature for 30 min was added dropwise to an ISFET gate electrode (hydrogen ion sensitive electrode 23). Thereafter, it was immersed in 5% glutaraldehyde, and left to stand at room temperature for 24 hrs. Accordingly, an immobilizing layer 51 was formed on the ISFET electrode. Then the ISFET electrode having thus formed immobilizing layer 51 (modified ISFET electrode) was immersed in a 5 mg/ml H⁺-pyrophosphatase solution, and left to stand at 4° C. for 24 hrs whereby immobilizing the H⁺-pyrophosphatase on the ISFET gate electrode. A sodium pyrophosphate solution was added to this H⁺-pyrophosphatase-immobilized ISFET electrode such that each final concentration of sodium pyrophosphate became 20 μM, 40 μM, 60 μM, 80 μM and 100 μM, respectively, to initiate the hydrolyzing reaction of the pyrophosphate by the H⁺-pyrophosphatase.

A result was obtained showing approximately straight-line relationship between the concentration of sodium pyrophosphate in the sample liquid, and the gate voltage value yielded when the voltage of 4.0 V was applied between the source and drain of the H⁺-pyrophosphatase-immobilized ISFET electrode using a silver/silver chloride electrode as a reference electrode 27 while keeping the electric current value between the source and drain of 400 μA. Accordingly, it was proven that the amount of pyrophosphate can be measured by the present method.

### EXAMPLE 3

This Example relates to manufacture of the pyrophosphate detection sensor according to the fifth embodiment.

First, vacuolar membranous H⁺-pyrophosphatase derived from seeds of pumpkin was purified similarly to Example 2 described above.

Next, 50 mg/ml vacuolar membranous H⁺-pyrophosphatase described above, 0.1 M pyrrol, and 1 M potassium chloride were admixed to give a mixture for electrolytic polymerization. In this mixture for electrolytic polymerization were immersed a platinum electrode that is a polarizable electrode 81 and a silver/silver chloride electrode as a reference electrode 27. Constant voltage of +1 V was applied to the platinum electrode for 6 min to yield a H⁺-pyrophosphatase-immobilized polypyrrol membrane-modified electrode by electrolytic polymerization. A sodium pyrophosphate solution was added to thus resulting H⁺-pyrophosphatase-immobilized polypyrrol membrane-modified electrode such that each final concentration of sodium pyrophosphate became 20 μM, 40 μM, 60 μM, 80 μM and 100 μM, respectively, to initiate the hydrolyzing reaction of pyrophosphate by the H⁺-pyrophosphatase.

A result was obtained showing approximately straight-line relationship between the concentration of sodium pyrophosphate, and the electric current indicated by the H⁺-pyrophosphatase-immobilized polypyrrol membrane-modified electrode when the electric potential of 300 mV was applied using a silver/silver chloride electrode as a reference electrode 27. Accordingly, it was proven that the amount of pyrophosphate can be measured by the present method.

### EXAMPLE 4

In this Example, detection of λDNA (regarding the entire base sequence of λDNA, see, GenBank database Accession Nos. V00636, J02459, M17233, X00906) in a sample was carried out.

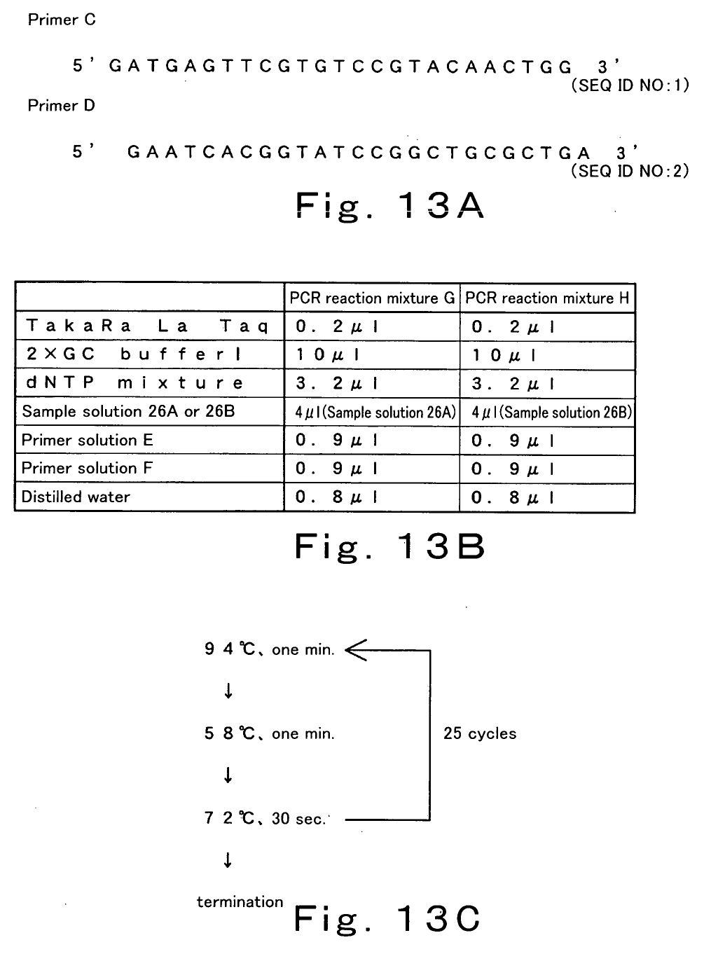

First, a sample solution 26A containing λDNA (manufactured by Takara Shuzo Co., Ltd.) dissolved in distilled water at a concentration of 10 ng/μL, and a sample solution 26B consisting of distilled water alone were provided. Further, as shown in FIG. 13A, primer solutions E and F (both 20 μM) containing two kinds of primer C (SEQ ID NO: 1) and primer D (SEQ ID NO: 2) dissolved in distilled water, respectively, which can completely hybridize to a specified base sequence of λDNA were provided.

To each of the sample solution 26A and 26B, were added TaKaRa La Taq (5 U/μL, manufactured by Takara Shuzo Co., Ltd.), a 2×GC buffer I that is an exclusive buffer for TaKaRa La Taq (manufactured by Takara Shuzo Co., Ltd.), a dNTP mixture (each concentration being 2.5 mM, manufactured by Takara Shuzo Co., Ltd.), and primer solutions E and F to prepare the PCR reaction mixtures G and H having the composition shown in FIG. 13B.

Next, for the PCR reaction mixtures G and H, a PCR reaction was carried out under the reaction temperature condition shown in FIG. 13C.

After termination of the PCR reaction, each of the PCR reaction mixtures G and H was subjected to the measurement of the electric current using the H⁺-pyrophosphatase electrode described in the above Example 1 through applying an electric potential of 200 mV with a silver/silver chloride electrode as a reference electrode similarly to Example 1. Accordingly, the PCR reaction mixture G exhibited evidently greater electric current value than the PCR reaction mixture H. In other words, it was proven that in the PCR reaction mixture G, a primer extension reaction proceeded. Therefore, it was revealed that a target nucleic acid can be detected according to the present method.

### EXAMPLE 5

In this Example, a variant λDNA was produced by artificially substituting a certain base, which is present in the base sequence of λDNA, with other base. Possible discrimination between normal λDNA and the variant λDNA was then investigated.

First, a variant λDNA (SEQ ID NO: 4) was produced using XDNA (manufactured by Takara Shuzo Co., Ltd.) (SEQ ID NO: 3). The variant λDNA was obtained by artificially substituting a GC base pair (region R₁ in the Figure), which exists within the double stranded DNA of the λDNA (hereinafter, normal ADNA is referred to as wild type λDNA), with an AT base pair (region R₂ in the Figure) by a method known to persons skilled in the art.

Next, the wild type λDNA and the variant λDNA were dissolved in distilled water to give the final concentration of 10 ng/μL, respectively, whereby preparing a wild type λDNA liquid and a variant λDNA liquid, respectively.

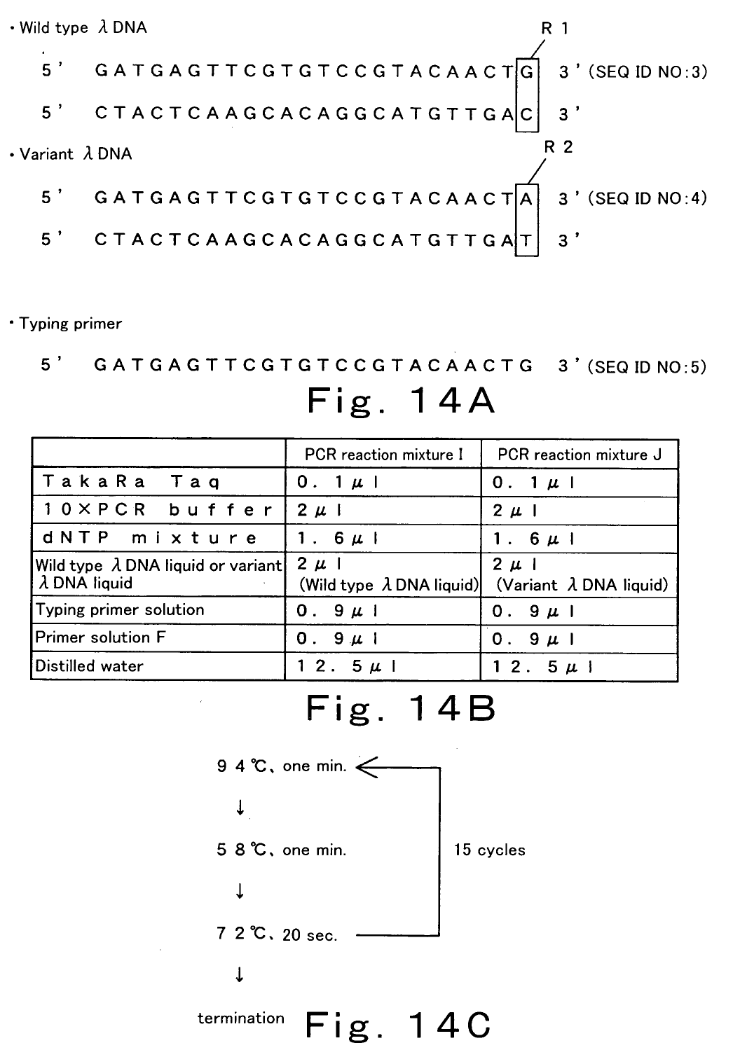

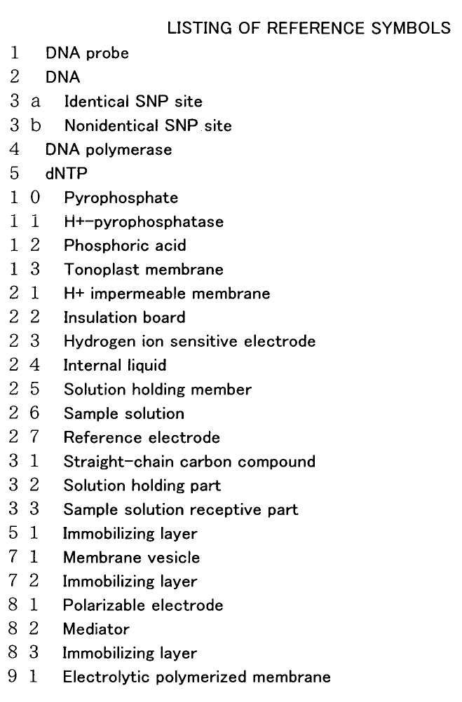

Then, for discriminating the difference in the base described above, a typing primer shown in FIG. 14A (SEQ ID NO: 5) was provided. Subsequently, a typing primer solution was provided by dissolving the typing primer to give the final concentration of 20 μM in distilled water.

The typing primer shown in FIG. 14A completely hybridizes to a single stranded DNA which is denoted in the bottom column of the wild type λDNA. However, “G” at the 3′ end of this typing primer can not hybridize to the single stranded DNA denoted in the bottom column of the variant λDNA. Therefore, when a primer extension reaction is carried out using this typing primer, the reaction favorably proceeds in case of the wild type λDNA, however, the reaction does not proceed so well in case of the variant λDNA.

Moreover, the primer solution F which was used in Example 4 described above was also provided.

Next, for each of the wild type λDNA liquid and the variant λDNA liquid, PCR reaction mixtures I and J having the composition shown in FIG. 14B were prepared using TaKaRa Taq (5 U/μL, manufactured by Takara Shuzo Co., Ltd.), a 10×PCR buffer that is exclusive for TaKaRa Taq (manufactured by Takara Shuzo Co., Ltd.), and a dNTP mixture (each concentration: 2.5 mM, manufactured by Takara Shuzo Co., Ltd.), and the typing primer solution and the primer solution F.

Then, for the PCR reaction mixtures I and J, a PCR reaction was carried out under the reaction temperature condition shown in FIG. 14C.

After termination of the PCR reaction, each of the PCR reaction mixtures was introduced to the H⁺-pyrophosphatase-immobilized modified ISFET electrode. The modified ISFET electrode is similar to that used in Example 2 described above.

Using this modified ISFET electrode, gate voltage was measured when the voltage of 4.0 V was applied between the source and drain of the H⁺-pyrophosphatase-immobilized ISFET electrode using a silver/silver chloride electrode as a reference electrode while keeping the electric current value between the source and drain of 400 μA, similarly to Example 2. Accordingly, the PCR reaction mixture I exhibited evidently greater voltage value than the PCR reaction mixture J. In other words, it was proven that in the PCR reaction mixture I, a primer extension reaction proceeded. It is believed that this is responsible for transport of the hydrogen ion to the side of the modified ISFET electrode resulting from the one base extension reaction caused by dATP in the extension reaction mixture I which contains the variant λDNA, consequently leading to a reaction between the produced pyrophosphate and the H⁺-pyrophosphatase on the modified ISFET electrode, while no extension reaction was caused in the reaction mixture J.

Therefore, according to this Example, it was revealed that the difference in one base pair in a specified base sequence of a DNA can be discriminated. In other words, this Example suggests that the method of the present invention is extremely advantageous in discriminating specified base sequences such as discrimination of the base type at a SNP site, mutation of one base pair due to discontinuous variation, and the like.

From the foregoing description, numerous improvements and other embodiment of the present invention would be apparent to persons skilled in the art. Therefore, the above description should be construed as merely an exemplification, which was provided for the purpose of teaching the persons skilled in the art the best embodiment for carrying out the present invention. Details of the structure and/or function can be substantially altered without departing from the spirit of the present invention.

### INDUSTRIAL APPLICABILITY

The pyrophosphate detection sensor according to the present invention can be utilized in, for example, discrimination of the base type of a SNP site, and thus, it is useful in tailor-made therapies such as drug administration on the basis of SNP typing. Also, the pyrophosphate detection sensor according to the present invention is useful in analysis of discontinuous variation in a base sequence of a DNA, and the results of such analysis can be utilized in drug discovery and clinical applications.

In addition, the pyrophosphate detection sensor according to the present invention can be utilized for detecting a nucleic acid having a specified base sequence, and the detection of a nucleic acid is useful in diagnoses of hereditary diseases, contamination monitoring of foods with bacteria, viruses and the like, and tests for infection in human bodies.

### FREE TEXT OF SEQUENCE LISTINGS

<223> of SEQ ID NO: 1: primer
<223> of SEQ ID NO: 2: primer
<223> of SEQ ID NO: 3: double stranded DNA
<223> of SEQ ID NO: 4: double stranded DNA
<223> of SEQ ID NO: 5: primer

1. A pyrophosphate detection sensor for detecting pyrophosphate in a sample solution which comprises:
a sample solution receptive part that receives said sample solution,
a H⁺ impermeable membrane having H⁺-pyrophosphatase,
an immobilizing layer for immobilizing said H⁺ impermeable membrane, and
a measurement means for measuring an electrochemical alteration with the change in hydrogen ion concentration of said immobilizing layer,
wherein said H⁺-pyrophosphatase is provided such that it hydrolyzes the pyrophosphate in said sample solution to result in change in the hydrogen ion concentration of the immobilizing layer.

2. The pyrophosphate detection sensor according to claim 1 wherein said immobilizing layer immobilizes the H⁺ impermeable membrane on the upper face or inside thereof.

3. The pyrophosphate detection sensor according to claim 1 wherein said H⁺ impermeable membrane is a membrane vesicle, and said immobilizing layer immobilizes said H⁺ impermeable membrane inside thereof.

4. The pyrophosphate detection sensor according to claim 1 wherein said measurement means has a hydrogen ion sensitive electrode which is in contact with said immobilizing layer, and a reference electrode provided such that it is in contact with said sample solution in the state of receiving said sample solution.

5. The pyrophosphate detection sensor according to claim 4 wherein said measurement means measures an alteration of the electric potential difference between said hydrogen ion sensitive electrode and said reference electrode.

6. The pyrophosphate detection sensor according to claim 4 wherein said immobilizing layer comprises a polymer gel or a self-assembled monomolecular membrane.

7. The pyrophosphate detection sensor according to claim 1 wherein said immobilizing layer comprises a material which causes an oxidation reduction reaction with change in the hydrogen ion concentration, and
said measurement means has a polarizable electrode that is in contact with said immobilizing layer, and a reference electrode provided such that it is in contact with said sample solution in the state of receiving said sample solution.

8. The pyrophosphate detection sensor according to claim 7 wherein said measurement means measures an alteration of the electric current between said polarizable electrode and said reference electrode.

9. The pyrophosphate detection sensor according to claim 7 wherein said immobilizing layer comprises a polymer gel containing a mediator which causes an oxidation reduction reaction with change in the hydrogen ion concentration, or a self-assembled monomolecular membrane, and said H⁺ impermeable membrane is immobilized on the upper face thereof.

10. The pyrophosphate detection sensor according to claim 7 wherein said immobilizing layer comprises an electrolytic polymer material which causes an oxidation reduction reaction with change in the hydrogen ion concentration.

11. A method of the detection of a nucleic acid having a specified base sequence wherein the pyrophosphate detection sensor according to claim 1 is used,
said method comprising the steps of:
(a) preparing a sample solution comprising a sample, a primer having a base sequence including a complementary binding region which complementarily binds to said nucleic acid, and a nucleotide;
(b) subjecting said sample solution to a condition which causes an extension reaction of said primer, and producing pyrophosphate when said extension reaction is caused;
(c) making a state in which said sample solution is received in said sample solution receptive part of said pyrophosphate detection sensor,
(d) measuring an electrochemical alteration with the change in hydrogen ion concentration of said immobilizing layer by said measurement means of said pyrophosphate detection sensor;
(e) detecting said extension reaction on the basis of the measurement result in the step (d), and
(f) detecting said nucleic acid on the basis of the detection result in the step (e).

12. A method of the discrimination of a base type in a base sequence of a nucleic acid wherein the pyrophosphate detection sensor according to claim 1 is used,
said method comprising the steps of:
(a) preparing a sample solution comprising a nucleic acid, a primer having a base sequence including a complementary binding region which complementarily binds to said nucleic acid, and a nucleotide;
(b) subjecting said sample solution to a condition which causes an extension reaction of said primer, and producing pyrophosphate when said extension reaction is caused;
(c) making a state in which said sample solution is received in said sample solution receptive part of said pyrophosphate detection sensor,
(d) measuring an electrochemical alteration with the change in hydrogen ion concentration of said immobilizing layer by said measurement means of said pyrophosphate detection sensor;
(e) detecting said extension reaction on the basis of the measurement result in the step (d), and
(f) discriminating the base type in the base sequence of said nucleic acid on the basis of the detection result in the step (e).


SEQUENCE LISTING (5 sequences)

SEQ ID NO: 1 - DNA, 25 bp, Artificial Sequence
  Oligonucleotide or Synthetic Sequence
gatgagttcgtgtccgtacaactgg

SEQ ID NO: 2 - DNA, 25 bp, Artificial Sequence
  Oligonucleotide or Synthetic Sequence
gaatcacggtatccggctgcgctga

SEQ ID NO: 3 - DNA, 24 bp, Lamda phage
  misc_feature: Double stranded
gatgagttcgtgtccgtacaactg

SEQ ID NO: 4 - DNA, 24 bp, Artificial Sequence
  Oligonucleotide or Synthetic Sequence
gatgagttcgtgtccgtacaacta

SEQ ID NO: 5 - DNA, 24 bp, Artificial Sequence
  Oligonucleotide or Synthetic Sequence
gatgagttcgtgtccgtacaactg